(19) United States
(12) Patent Application Publication
(10) Pub. No.: US 2019/0172593 A1
(43) Pub. Date: Jun. 6, 2019

(54) SYSTEM AND METHOD FOR STORING AND DELIVERING HEALTHCARE INFORMATICS DATA

(71) Applicant: RANDEEP SINGH, Pitampur (IN)

(72) Inventor: RANDEEP SINGH, Pitampur (IN)

(21) Appl. No.: 16/268,575

(22) Filed: Feb. 6, 2019

Related U.S. Application Data
(63) Continuation-in-part of application No. 15/741,747, filed on Jan. 3, 2018.

(30) Foreign Application Priority Data
Jan. 6, 2016 (IN) 3630/DEL/2015

(51) Int. Cl. G16H 80/00; G16H 10/60

(52) U.S. Cl. CPC G16H 80/00; G16H 50/20; G16H 10/60

(57) ABSTRACT

Tagged writing receivers, such as surface-based devices, include identity codes that identify the tagged writing receiver. The tagged writing receiver has content written on it in real-time and digitally saved in real-time. An addendum code can be activated by touching a sub-zone of the receiver with a tagged writing instrument to save the content in the sub-zone in a particular authentication database at a network destination, such as a hospital or treatment center. The tagged writing instrument writes on the tagged writing receiver and also captures the content from the tagged writing receiver. The tagged writing instrument also has identity codes that identify the tagged writing instrument. The tagged writing instrument converts the written content into recognized data items for saving.

### RELATED APPLICATIONS

This application claims priority under 35 U.S.C. § 120 to, and is a continuation-in-part of, co-pending application 15/741,747, filed Jan. 3, 2018, which is a National Stage entry of International Application PCT/IN2017/050008, filed Jan. 6, 2017, which claims priority to Indian Application 3630/DEL/2015, filed Jan. 6, 2016. These co-pending, Indian, and International applications are incorporated by reference herein in their entireties.

### FIELD

This invention relates to the field of medical engineering and computer networks.

Particularly, this invention relates to a system and method for storing and delivering healthcare informatics data.

Specifically, this invention relates to a system and method for controlled access, distribution, delivery, and consumption of data pertaining to doctor-patient interaction.

### BACKGROUND

Healthcare data management is a very important aspect of managing and caring for the personal health of an individual. Over a course of time, an individual is susceptible to fall sick, a plurality of times, and it is vital for the individual to manage and store details of history of illnesses, diseases, and treatments.

However, it is often a challenge to store this healthcare information in an easily accessible manner and in a safe location.

Clinicians and doctors usually type, write, or speak-out prescriptions and treatment plans for a patient. While the spoken information is mostly for immediate benefit and information of the patient, the written or typed medical records are kept for long-term storage and future references. However, the medical records remain with clinicians or hospitals and patients do not have access to original records, always. In times of emergencies or change in treatment plans, the records are to be shared between different health care providers such as doctors or hospitals for effective treatment. Sharing of information between the various health care providers is not easy, as the information is not readily accessible and not available to the patient.

Patients sometimes request hospitals or doctors to share a physical copy or a digitized copy of the healthcare records. However, it is very difficult for a patient to store all the healthcare information in an integrated and methodical manner. Since there is no uniformity in the quality or the type of healthcare information shared by a hospital or a doctor, the healthcare information of an individual are often stored in a plurality of locations. This fragmented and often incomplete storage of information results in a lack of proper information about an individual's medical history.

Hence, there is a need for storing and delivering healthcare informatics data in an integrated and uniform manner. There is also a need for digitizing printed and handwritten healthcare information to provide an integrated approach to storage and delivery of healthcare information.

The above-mentioned shortcomings, disadvantages, and problems are addressed herein and which will be understood by reading and studying the following specification.

Each year, millions of faxes still flow into the practices of the more than hundreds of thousands of service providers during a doctor-patient interaction. In healthcare, faxes remain the most common method that practitioners use to communicate with each other, and therefore often contain important clinical information: lab results, specialist consult notes, prescriptions, diagnosis, prognosis, medications, referrals, and the like. Faxes do not contain any structured text; so, it takes medical practice staff an average of 2 minutes and 36 seconds to review each document and input relevant data into patient records. A combination of machine learning and business-process outsourcing has automated categorizing of faxes and, therefore, reduced this quantum of time to over a minute. This ‘over-a-minute’ time required is still unaddressed latency in the system and it still requires a two-step procedure.

But, this is still a cumbersome process since it still requires at least a two-step procedure where, in a first step, a doctor writes on a prescription pad and then, in a second step, faxes this information. This is not seamless experience.

There is a need for a system and method which eliminates this latency and which simultaneously eliminates the millions of hours of work from the healthcare system which still exists. The system and method of this invention provides a solution towards eliminating faxes altogether; thereby, not only saving operational costs but also saving capital costs.

### SUMMARY

An object of the invention is to provide a system and method for displaying, storing, and delivering healthcare informatics data.

Another object of the invention is to provide a system and method for controlled display, access, distribution, delivery, and consumption of healthcare informatics data.

Yet another object of the invention is to provide a system and method for digitizing printed and handwritten healthcare information, such as prescription and patient case history, and contextually delivering and displaying the digitized information.

Still another object of the invention is to provide a healthcare informatics system that puts the patients at the center of healthcare and in control of data generated about their particular medical condition.

An additional object of the invention is to provide a healthcare informatics system that is very intuitive and easy to use for healthcare providers and relevant stakeholders such as clinicians, nurses, pharmacists, hospitals and diagnostics tests providers.

These and other objects and advantages of the embodiments herein will become readily apparent from the following summary and the detailed description taken in conjunction with the accompanying drawings.

According to this invention, there is provided a method for storing and delivering healthcare informatics data within a network of nodes, the method comprising the steps of:
providing a tagged writing surface embedded with a first code configured to provide an identity to the tagged writing surface, the tagged writing surface configured in a manner that it captures content written on it, in real-time, and transmits it to a digital medium in real-time;
providing a tagged writing apparatus configured to write on the tagged writing surface and further configured to, concurrently, capture the written content from the tagged writing surface, the tagged writing apparatus being embedded with a second code configured to provide an identity to the tagged writing apparatus, the tagged writing apparatus configured to convert at least some of the written content into recognized data items;
providing at least an addendum code configured to be activated by the tagged writing apparatus, the addendum code being activated only by a correlative tagged writing apparatus, the addendum code being pre-configured to be correlated with a pre-defined zone of the tagged writing surface wherein, upon its activation, the tagged writing surface realizes the zone along with data items in the zone and stores it in a fourth database, the fourth database being communicably coupled to an authentication mechanism which limits access of information of the zone coupled with the addendum code to communicably coupled nodes as defined by the rule engine;
   characterized in that, the tagged writing zone comprising pre-defined zones, each pre-defined zone being configured with the addendum code in a correlational manner such that each addendum code and, therefore, its associated zone is correlative to an identified node in the network in such a manner that recognized data items from the written content from a pre-defined zone of the tagged writing surface, in association with its addendum code, is configured by a rule engine to behave in accordance with pre-defined rules of the rule engine, thereby providing rule-linked addendum codes, associated with a node in the network, for pre-defined portions of the tagged writing surface;

Typically, the first code, along with its correlation with the tagged writing surface, is stored in a correlational manner in a first database.

Typically, the second code, along with its correlation with the tagged writing apparatus, is stored in a correlational manner in a second database.

Typically, the tagged writing surface is configured with pre-determined zones or pixels or defined areas which are recognized by the first code and the tagged writing apparatus is configured to determine which portion of the tagged writing surface is being written upon based on these pre-determined zones or pixels or defined areas.

Typically, the tagged writing surface comprises a first code which identifies itself with a doctor using the tagged writing surface or a clinic or hospital that issues the tagged writing surface.

Typically, the tagged writing apparatus comprises a second code which identifies itself with a doctor using the writing surface or a clinic or hospital that issues the tagged writing apparatus.

Typically, the step of providing a tagged writing apparatus comprises a step of providing an imaging device along with a communication module, characterized in that, the imaging device is configured to capture and convert any data, which is physically written, using the tagged writing apparatus into digital data and the communication module is configured to transmit the digital data to other nodes.

Typically, the tagged writing surface is a physical sheet customized to pen information.

Typically, the first code of the tagged writing surface correlates with at least a set of static data items, stored in a database, the static data items comprising at least a set of preset customized static data items selected from a group of data items consisting of doctor information related data items, clinic related data items, hospital information related data items, and tag information related data items.

Typically, the first code of the tagged writing surface correlates with at least a set of dynamic data items, stored in a database, the dynamic data items comprising at least a set of dynamic information that is written on the tagged writing surface, the dynamic information comprises a set of dynamic data items selected from a group of data items consisting of patient's name related data items, patient's demographics related data items, patient's identity related data items, patient's illness related data items, patient's treatment plan related data items, prescription related data items, date of doctor-patient interaction related data items, time of doctor-patient interaction related data items, and location of doctor-patient interaction related data items.

Typically, the first code, the second code, and the addendum code is selected from a group of codes consisting of a QR codes, bar codes, and RFID codes.

Typically, method comprises a step of collating all information data items, per doctor-patient interaction, and stores it as a unified dataset along with date stamp and time stamp in a third database.

Typically, the step of providing a tagged writing surface is provided with a configuration step for configured the tagged writing surface in a manner that it recognizes a doctor's handwritten transcription, in real-time and transmits a transliterated version of the handwritten transcription to a digital medium in real-time, thereby transcribing a doctor's handwritten transcription into a digitally recognized font.

Typically, the step of providing a tagged writing surface is provided with a configuration step for configured the tagged writing surface in a manner that it recognizes a doctor's handwritten transcription, in real-time and transmits a translated version of the handwritten transcription to a digital medium in real-time thereby transcribing a doctor's handwritten transcription into a digitally recognized font. Typically, the tagged writing surface is communicably coupled to an intermittent display mechanism which displays information on the tagged writing surface, in real time, within the boundaries of the tagged writing surface.

Typically, the network of nodes, comprising at least a nodes selected from a group of nodes consisting of doctor nodes, patient nodes, care giver nodes, hospital nodes, clinic nodes, pharmacy nodes, laboratory nodes, specialist nodes, diagnostic center nodes, test center nodes, payment nodes, insurance nodes, and administrator nodes.

Typically, the method comprises a step of tagging per clinic or per hospital or per doctor, the tagging being configured to correlate at least one of the following:
a) a doctor identity with a first code and a corresponding tagged writing surface;
b) a doctor identity with a second code and a corresponding tagged writing apparatus;
c) a patient identity with a first code and a corresponding tagged writing surface;
d) a patient identity with a second code and a corresponding tagged writing apparatus;
e) a hospital identity or a clinic identity with a first code and a corresponding tagged writing surface;
f) a hospital identity or a clinic identity with a second code and a corresponding tagged writing apparatus.

Typically, the method comprising a step of tagging configured per clinic or per hospital or per doctor, the tagging further comprising a step of allowing simultaneous reading of the first code and the second code, the codes being read by an application that allows for associating codes with each other or codes with identities, the tagging station further being associated with the rule engine in order to define rules for each association.

Typically, the method comprises a step of tagging configured per clinic or per hospital or per doctor, the step of tagging further comprising a step of allowing simultaneous reading of the first code and the addendum code, the codes being read by an application that allows for associating codes with each other or codes with identities, the tagging station further being associated with the rule engine in order to define rules for each association.

Typically, the method providing a step of providing weighted and mapped pixels on the tagged writing surface, the map of each pixel is its absolute position on the tagged writing surface along with its relative position in terms of various zones on the tagged writing surface, the weight of each pixel defining an origin node associated with that pixel (and, hence, the contoured zone which carries that pixel) along with a destination node associated with that pixel (and, hence, the contoured zone which carries that pixel), thereby translating a previously static tagged writing surface into a now dynamic tagged writing surface.

Typically, the method further comprises a step of contouring zones or outlines of zones drawn on the tagged writing surface to read pixel weight and map within each contour and display it virtually after which a user, then, uses this contoured virtual display to associate it with an addendum code and processes to define an origin node, destination node, and the like nodes per pixel or per zone (group of pixels).

Typically, the method comprising a step of storage of identification vectors of a doctor's signature and the system further comprising a comparator to compare the doctor's real-time signature in terms of the identification vectors in order to authenticate data on the tagged writing surface using the tagged writing apparatus used to write and capture data.

Typically, the system comprising a step of providing at least an addendum code configured to be activated by the tagged writing apparatus, the addendum code being activated only by a correlative tagged writing apparatus, the addendum code being pre-configured to be correlated with a pre-defined zone of the tagged writing surface wherein, upon its activation, the tagged writing surface realizes the zone along with data items in the zone and stores it in a third database, in a unified manner.

According to this invention, there is provided a system for storing and delivering healthcare informatics data within a network of nodes, the system comprising:
a tagged writing surface embedded with a first code configured to provide an identity to the tagged writing surface, the tagged writing surface configured in a manner that it captures content written on it, in real-time, and transmits it to a digital medium in real-time;
a tagged writing apparatus configured to write on the tagged writing surface and further configured to, concurrently, capture the written content from the tagged writing surface, the tagged writing apparatus being embedded with a second code configured to provide an identity to the tagged writing apparatus, the tagged writing apparatus configured to convert at least some of the written content into recognized data items;
at least an addendum code configured to be activated by the tagged writing apparatus, the addendum code being activated only by a correlative tagged writing apparatus, the addendum code being pre-configured to be correlated with a pre-defined zone of the tagged writing surface wherein, upon its activation, the tagged writing surface realizes the zone along with data items in the zone and stores it in a fourth database, the fourth database being communicably coupled to an authentication mechanism which limits access of information of the zone coupled with the addendum code to communicably coupled nodes as defined by the rule engine;
   characterized in that, the tagged writing zone comprising pre-defined zones, each pre-defined zone being configured with the addendum code in a correlational manner such that each addendum code and, therefore, its associated zone is correlative to an identified node in the network in such a manner that recognized data items from the written content from a pre-defined zone of the tagged writing surface, in association with its addendum code, is configured by a rule engine to behave in accordance with pre-defined rules of the rule engine, thereby providing rule-linked addendum codes, associated with a node in the network, for pre-defined portions of the tagged writing surface;

Typically, the first code, along with its correlation with the tagged writing surface, is stored in a correlational manner in a first database.

Typically, the second code, along with its correlation with the tagged writing apparatus, is stored in a correlational manner in a second database.

Typically, the tagged writing surface is configured with pre-determined zones or pixels or defined areas which are recognized by the first code and the tagged writing apparatus is configured to determine which portion of the tagged writing surface is being written upon based on these pre-determined zones or pixels or defined areas.

Typically, the tagged writing surface comprises a first code which identifies itself with a doctor using the tagged writing surface or a clinic or hospital that issues the tagged writing surface.

Typically, the tagged writing apparatus comprises a second code which identifies itself with a doctor using the writing surface or a clinic or hospital that issues the tagged writing apparatus.

Typically, the tagged writing apparatus comprises an imaging device along with a communication module, characterized in that, the imaging device is configured to capture and convert any data, which is physically written, using the tagged writing apparatus into digital data and the communication module is configured to transmit the digital data to other nodes.

Typically, the tagged writing surface is a physical sheet customized to pen information.

Typically, the first code of the tagged writing surface correlates with at least a set of static data items, stored in a database, the static data items comprising at least a set of preset customized static data items selected from a group of data items consisting of doctor information related data items, clinic related data items, hospital information related data items, and tag information related data items.

Typically, the first code of the tagged writing surface correlates with at least a set of dynamic data items, stored in a database, the dynamic data items comprising at least a set of dynamic information that is written on the tagged writing surface, the dynamic information comprises a set of dynamic data items selected from a group of data items consisting of patient's name related data items, patient's demographics related data items, patient's identity related data items, patient's illness related data items, patient's treatment plan related data items, prescription related data items, date of doctor-patient interaction related data items, time of doctor-patient interaction related data items, and location of doctor-patient interaction related data items.

Typically, the first code, the second code, and the addendum code is selected from a group of codes consisting of a QR codes, bar codes, and RFID codes.

Typically, a collator collates all information data items, per doctor-patient interaction, and stores it as a unified dataset along with date stamp and time stamp in a third database.

Typically, the tagged writing surface is configured in a manner that it recognizes a doctor's handwritten transcription, in real-time and transmits a transliterated version of the handwritten transcription to a digital medium in real-time, thereby transcribing a doctor's handwritten transcription into a digitally recognized font.

Typically, the tagged writing surface is configured in a manner that it recognizes a doctor's handwritten transcription, in real-time and transmits a translated version of the handwritten transcription to a digital medium in real-time thereby transcribing a doctor's handwritten transcription into a digitally recognized font.

Typically, the tagged writing surface is communicably coupled to an intermittent display mechanism which displays information on the tagged writing surface, in real time, within the boundaries of the tagged writing surface.

Typically, the network of nodes, comprising at least a nodes selected from a group of nodes consisting of doctor nodes, patient nodes, care giver nodes, hospital nodes, clinic nodes, pharmacy nodes, laboratory nodes, specialist nodes, diagnostic center nodes, test center nodes, payment nodes, insurance nodes, and administrator nodes.

Typically, the system comprises a tagging station configured per clinic or per hospital or per doctor, the tagging station being configured to correlate at least one of the following:
a) a doctor identity with a first code and a corresponding tagged writing surface;
b) a doctor identity with a second code and a corresponding tagged writing apparatus;
c) a patient identity with a first code and a corresponding tagged writing surface;
d) a patient identity with a second code and a corresponding tagged writing apparatus;
e) a hospital identity or a clinic identity with a first code and a corresponding tagged writing surface;
f) a hospital identity or a clinic identity with a second code and a corresponding tagged writing apparatus.

Typically, the system comprises a tagging station configured per clinic or per hospital or per doctor, the tagging station further comprising an aperture that allows simultaneous reading of the first code and the second code, the codes being read by an application that allows for associating codes with each other or codes with identities, the tagging station further being associated with the rule engine in order to define rules for each association.

Typically, the system comprises a tagging station configured per clinic or per hospital or per doctor, the tagging station further comprising an aperture that allows simultaneous reading of the first code and the addendum code, the codes being read by an application that allows for associating codes with each other or codes with identities, the tagging station further being associated with the rule engine in order to define rules for each association.

Typically, the system comprising a processor configured to provide weights and maps to each pixels on the tagged writing surface, the map of each pixel is its absolute position on the tagged writing surface along with its relative position in terms of various zones on the tagged writing surface, the weight of each pixel defining an origin node associated with that pixel (and, hence, the contoured zone which carries that pixel) along with a destination node associated with that pixel (and, hence, the contoured zone which carries that pixel), thereby translating a previously static tagged writing surface into a now dynamic tagged writing surface.

Typically, the system further comprises a contourer, coupled with a processor, for contouring zones or outlines of zones drawn on the tagged writing surface to read pixel weight and map within each contour and display it virtually after which a user, then, uses this contoured virtual display to associate it with an addendum code and processes to define an origin node, destination node, and the like nodes per pixel or per zone (group of pixels).

Typically, the system comprising storage of identification vectors of a doctor's signature and the system further comprising a comparator to compare the doctor's real-time signature in terms of the identification vectors in order to authenticate data on the tagged writing surface using the tagged writing apparatus used to write and capture data.

Typically, the system comprising at least an addendum code configured to be activated by the tagged writing apparatus, the addendum code being activated only by a correlative tagged writing apparatus, the addendum code being pre-configured to be correlated with a pre-defined zone of the tagged writing surface wherein, upon its activation, the tagged writing surface realizes the zone along with data items in the zone and stores it in a third database, in a unified manner.

### BRIEF DESCRIPTION OF THE DRAWINGS:

The invention will now be described in relation to the accompanying drawings, in which:

FIG. 1 illustrates a schematic block diagram of the system;

FIG. 2 illustrates a block diagram of a healthcare information management system, according to an embodiment herein;

FIG. 3 illustrates a plan view of a sample (specimen) prescription paper with a pre-printed barcode at the top, according to an embodiment herein;

FIG. 4 illustrates a plan view of sample (specimen) prescription paper with a preprinted QR code at bottom, according to an embodiment herein;

FIG. 5 illustrates a block diagram of an online platform/portal for data sharing and consumption in a healthcare information management system, according to an embodiment herein;

FIG. 6 illustrates a system for data sharing and consumption in a healthcare information management system using digital pen and paper, according to an embodiment herein; and

FIG. 7 illustrates a flow chart explaining a process for data sharing and consumption in a healthcare information management system using digital pen and paper, according to an embodiment herein.

### DETAILED DESCRIPTION

Although the specific features of the embodiments herein are shown in some drawings and not in others. This is done for convenience only as each feature may be combined with any or all of the other features in accordance with the embodiment herein.

The term, ‘healthcare network’, or ‘healthcare’, generically refers to various personnel such as doctors, general practitioners, surgeons, specialist doctors, specialist surgeons, dentists, specialist dentists, physiotherapists, therapists, nurses, paramedical staff, nodes, systems, points of care, hospitals, clinics, dispensaries, nursing homes, imaging labs, diagnostic centers, test labs, testing labs, rehabilitation centers, operating rooms, recuperating centers, examination centers, chemists, pharmacies, ambulances, emergency units, and the like care-giving environments, and even insurance related practitioners and systems.

The system of this invention is configured to be used in a connected healthcare ecosystem with a plurality of nodes. Each node comprises a data transmission mechanism, a data reception mechanism, a data reader mechanism, a multimodal input mechanism, a multimodal output mechanism, a notification mechanism, a display mechanism, and a connector mechanism to enable one node connection with another node based on rules defined by an administrator of a network of nodes or a node owner. Thus, for a doctor-patient interaction or for a doctor-patient engagement, a network of nodes, in this connected ecosystem, may comprise some of the following nodes:
a) Doctor nodes;
b) Patient nodes;
c) Care giver nodes;
d) Hospital nodes;
e) Clinic nodes;
f) Pharmacy nodes;
g) Laboratory nodes;
h) Specialist nodes;
i) Diagnostic center nodes;
j) Test center nodes;
k) Payment nodes;
l) Insurance nodes;
m) Administrator nodes.

The various embodiments herein provide a system and method for storing and delivering healthcare informatics data within a healthcare network of nodes.

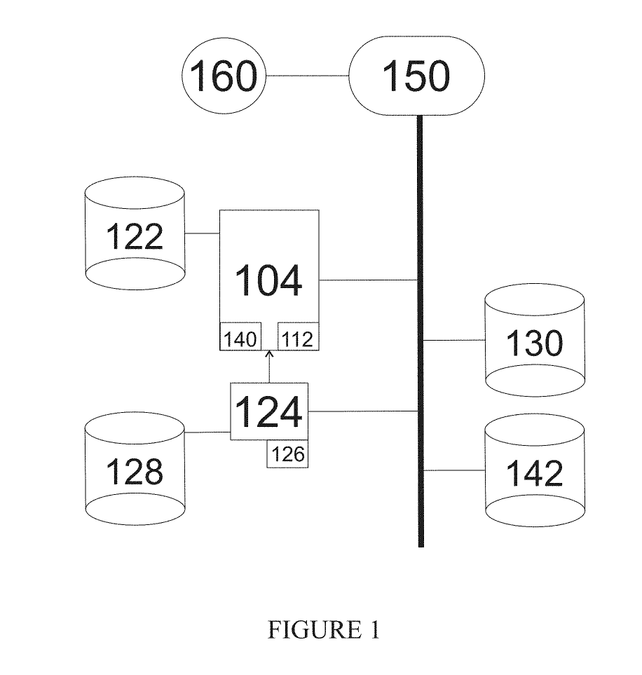

FIG. 1 illustrates a schematic block diagram of the system.

In at least an embodiment, there is provided a tagged writing surface (104). This tagged writing surface is embedded with a first code (112) configured to provide an identity to the tagged writing surface (104). This identity, along with its correlation with the tagged writing surface (104), is stored in a correlational manner in a first database (122).

In at least an embodiment, the tagged writing surface (104) is configured to be written upon by a tagged writing apparatus (124) configured to write on the tagged writing surface (104) and further configured to, simultaneously or concurrently, capture the written content from the tagged writing surface (104). The tagged writing apparatus (124) is embedded with a second code (126) configured to provide an identity to the tagged writing apparatus (124). This identity, along with its correlation with the tagged writing apparatus (126), is stored in a correlational manner in a second database (128).

The tagged writing surface (104) is configured with pre-defined zones or pixels or defined areas which are recognized by the first code (112). The tagged writing apparatus (124) is configured to determine which portion of the tagged writing surface (124) is being written upon based on these pre-defined zones or pixels or defined areas. Each pre-defined zone or each pixel or each defined area of the tagged writing surface is mapped, in terms of its location, on the tagged writing surface, and this mapping is stored in the first database along with its association with the first code. Thus, zone-code mapping and correlation or pixel-code mapping and correlation, area-code mapping and correlation is stored in the first database. Thus, while the tagged writing surface (124) can be drawn out into zones, the tagged writing surface (104) makes the system smarter by accurately recognizing these zones and capturing information correlative to the zones. A group of pixels can be bounded to form a zone for a purpose.

A display is employed in order to display the written matter on the tagged writing surface, in real-time.

In accordance with a non-limiting exemplary embodiment, the tagged writing surface (104) comprises a first code (112) which identifies itself with a doctor using the writing surface or a clinic or hospital that issues the tagged writing surface.

In accordance with a further non-limiting exemplary embodiment, the tagged writing apparatus (124) comprises a second code (126) which identifies itself with a doctor using the writing surface or a clinic or hospital that issues the tagged writing apparatus.

The tagged writing apparatus (124) comprises an imaging device along with a communication module. It may further comprise a memory module for storing data that is read/captured.

The imaging device is configured to capture and convert any data, which is physically written, using the writing apparatus into digital data. The communication module of the writing apparatus is configured to transmit the digital data to a server or a remote server or a cloud server for storage and further retrieval as and when necessary.

In at least an embodiment the tagged writing surface (104) is a physical sheet customized to pen information.

The first code of the tagged writing surface (104) correlates with at least a set of static data items, stored in a database. These data items comprise at least a set of preset customized static data items such as doctor information, clinic or hospital information, and tag information.

Additionally, the first code of the tagged writing surface (104) correlates with at least a set of dynamic data items, stored in a database. These data items comprise at least a set of dynamic information that is written on the tagged writing surface. This dynamic information comprises a set of data items such as patient's name, patient's demographics, patient's identity, patient's illness, patient's treatment plan, prescription, date of doctor-patient interaction, time of doctor-patient interaction, location of doctor-patient interaction, and the like.

The codes (tags) may be selected from a group of tags or codes consisting of a QR codes, bar codes, RFID codes, and the like codes.

Thus, for every doctor-patient interaction, the system first identifies the tagged writing surface along with the tagged writing apparatus. Then, the system identifies the preset static information. Further, the system starts identifying the customized information as and how the doctor transcribes on to the tagged writing surface (104) using the tagged writing apparatus (124). A collator collates all of this information, per doctor-patient interaction, and stores it as a unified dataset along with date stamp and time stamp in a third database (130).

The tagged writing surface (124) is configured in a manner that it captures a doctor's transcription, in real-time, and transmits it to a digital medium in real-time.

Additionally, the tagged writing surface (124) is configured in a manner that it recognizes a doctor's handwritten transcription, in real-time and transmits a transliterated version of the handwritten transcription to a digital medium in real-time. Thus, a doctor's handwritten transcription gets converted into a digitally recognized font. In at least an embodiment, the digital medium may be a display.

More additionally, the tagged writing surface (104) is configured in a manner that it recognizes a doctor's handwritten transcription, in real-time and transmits a translated version of the handwritten transcription to a digital medium in real-time. Thus, a doctor's handwritten transcription gets converted into a translated digitally recognized font.

While the digital medium may be just a database which stores information, it may also include an intermittent display mechanism (communicably coupled to the tagged writing surface) which displays information on the tagged writing surface (104) within the boundaries of the tagged writing surface; in real-time.

In at least an embodiment, the tagged writing zone comprised pre-defined zones. Each zone is configured with an addendum code in a correlational manner such that each addendum code and, therefore, its associated zone is correlative to an identified node in the network in such a manner that recognized data items from a specific zone of the tagged writing surface in association with its addendum code is configured by a rule engine (150) to act or behave in accordance with pre-defined rules. This action or behavior or rule is selected from a group of actions or behaviors or rules comprising transmitting to those data items to a specified node, masking information to certain nodes, storing data items in a secure manner, and the like. This, the rule engine is used to create rule-linked addendum codes for pre-defined portions of the tagged writing surface so that these rule-linked addendum codes are associated with a node in a network concerning doctor-patient interaction.

The tagged writing surface has pre-defined zones (medicines, follow-ups) which are pre-aligned. When the tagged writing apparatus hits a specific pixel in a specific zone, associated rules are fired. According to a non-limiting exemplary embodiment, for a pre-defined zone with an associated addendum code which is identified with a pharmacy node, when the tagged writing apparatus strikes a pixel in that zone, a medical database node is activated for checking allergies or contra-indications and a pharmacy node is simultaneously notified of the recognized data items in that zone. For MRI, and other laboratory zone, the laboratory node is pre-notified. The patient, therefore, has a seamless engagement experience, where data, in a customized and secure fashion, travels to a node before the patient arrives at that node.

In at least a non-limiting exemplary embodiment, a pre-defined zone on the tagged writing surface (104) along with its unique rule-linked addendum code (140) is configured to be associated with a laboratory node in the network for conducting MRIs. A doctor (102) uses the tagged writing apparatus (124) with its code (126) to write on the pre-defined zone on the tagged writing surface (104) which has its unique rule-linked addendum code (140) and action on the data from this portion of the tagged writing surface behaves according to rules associated with the rule-linked addendum code (140). One such example may be that the doctor (104) prescribes an MRI for a patient. This prescription is either recognized by the system using natural language processing or by the presence of a pre-defined checkmark which is identifiable by the system. In this scenario, only data from this pre-defined zone concerning the MRI is given to the laboratory conducting the MRI scan. Also, data from other portions of the tagged writing surface may be given to the laboratory depending on how their associated rule-linked addendum codes are defined.

In at least an embodiment, the tagged writing surface comprises at least an addendum code (140) configured to be activated by the tagged writing apparatus (124). Typically, the addendum code may be latent in each first code and is activated only by a correlative tagged writing apparatus. The addendum code may be pre-configured to be correlated with a pre-defined zone or pixels or area of the tagged writing surface. Upon its activation, the tagged writing surface realizes the zone and the data in the zone and stores it in the third database, in a unified manner, along with storing it in a fourth database (142), in a stand-alone manner. This fourth database, further, is communicably coupled to an authentication mechanism which limits access of the information in the zone coupled with the addendum code to communicably coupled nodes of the ecosystem as defined either by the system or the hospital or the clinic or the doctor. The activation of this addendum code acts as at least a primary authenticator to the authentication mechanism.

In accordance with a non-limiting exemplary embodiment, the pre-defined zone along with the addendum code is correlated or communicably coupled to a laboratory node which tests a patient. Thus, when a doctor uses the tagged writing apparatus to activate the addendum on the tagged writing surface, the communicably coupled laboratory node is notified of the data in the pre-defined zone, thereby notifying the laboratory of the tests to be performed along with patient identity, doctor identity, and the like. This data also forms a part of the third database with the unified dataset.

In accordance with a further non-limiting exemplary embodiment, the pre-defined zone along with the addendum code is correlated or communicably coupled to a pharmacy node which dispenses medicines to a patient. Thus, when a doctor uses the tagged writing apparatus to activate the addendum on the tagged writing surface, the communicably coupled pharmacy node is notified of the data in the pre-defined zone, thereby notifying the pharmacy of the medicines to be dispensed along with patient identity, doctor identity, and the like. This data also forms a part of the third database with the unified dataset.

In at least an embodiment, the system comprises a tagging station (160) configured per clinic or per hospital or per doctor, the tagging station being configured to correlate at least one of the following:
a) a doctor identity with a first code and a corresponding tagged writing surface;
b) a doctor identity with a second code and a corresponding tagged writing apparatus;
c) a patient identity with a first code and a corresponding tagged writing surface;
d) a patient identity with a second code and a corresponding tagged writing apparatus;
e) a hospital identity or a clinic identity with a first code and a corresponding tagged writing surface;
f) a hospital identity or a clinic identity with a second code and a corresponding tagged writing apparatus.

An aperture in the tagging station allows easy reading of the first code and second code, simultaneously; these codes are read by a phone and an application through the phone allows for associating codes with each other or codes with identities. This tagging station may be associated with a rule engine in order to define rules for each association on the fly.

In at least an embodiment, the first code is provided by the system of this invention and the addendum code is provided by a doctor or a hospital or a clinic such that the tagging station is configured to embed this addendum code to the first code or the second code at the point of care.

In at least an embodiment, the administrator node is communicably coupled to a rule engine configured to define rules in terms of linking each of the codes to the any of the plurality of database. Further, the rule engine is also configured to define rules in terms of providing or limiting access, of the tagged writing surface, or portions thereof, to various nodes of the ecosystem. The rule engine, basically, defines access control channels with respect to various nodes of a healthcare ecosystem.

In at least an embodiment, the tagged writing surface (104) comprises a plurality of pixels which are weighted and mapped pixels. The map of each pixel is its absolute position on the tagged writing surface along with its relative position in terms of various zones on the tagged writing surface. The weight of each pixel defines the origin node associated with that pixel (and, hence, the contoured zone which carries that pixel) along with a destination node associated with that pixel (and, hence, the contoured zone which carries that pixel). The weight of the pixel (and, hence, the contoured zone which carries that pixel) translates into data packets which comprise data that is written (by the tagged writing apparatus) onto that pixel (and, hence, the contoured zone which carries that pixel) along with path for that data packet (in terms of origin node and destination node) so that the data penned in that zone is relayed to a node, seamlessly and securely (no other zones' data is relayed to the destination node if it is not mapped). This is useful in a non-limiting exemplary scenario, in which, a first zone may be defined as a prescription zone weighted with an origin node as a doctor's node and a destination node as a pharmacy node and a second zone may be defined as a tests' zone weighted with an origin node as a doctor's node and a destination node as a laboratory node and the doctor wants only the prescription data of the prescription zone to be sent to the pharmacy node (and not the laboratory node) and the doctor wants only the tests' data of the tests' zone to be sent to the laboratory node (and not the prescription zone). Thus, this mechanism and system provides a variety of mapped, weighted, and functionalized zones, thereby translating a previously static tagged writing surface into a now dynamic tagged writing surface of the current invention. This weight and map is pre-configured and associated with at least an addendum code. Together, the weight and map of each pixel define a function for that pixel and, hence, for its corresponding zone in correlation with the node associated with it. A contourer is provided which is defined in a processor associated with the tagging station. This contourer traces zones or outlines of zones drawn on the tagged writing surface. The contourer is configured to read pixel weight and map within each contour and display it virtually. A user, then, uses this contoured virtual display to associate it with an addendum code or a plurality of addendum code. The processor, along with the contourer, can also be used to define origin node, destination node, and the like nodes per pixel or per zone (group of pixels).

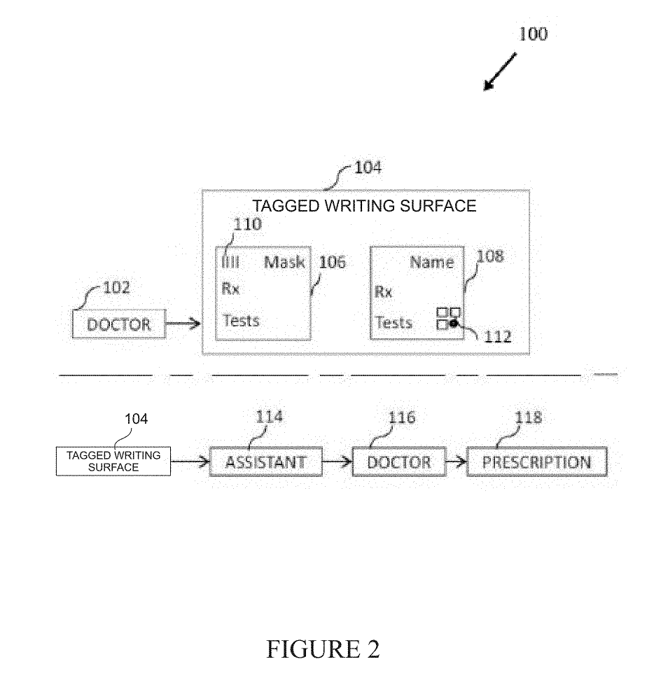

FIG. 2 illustrates an environment, where the embodiments herein are practiced. The environment 100 comprises a doctor 102, who writes on the tagged writing surface 104 of a first type 106, or a second type 108 upon seeing a patient, and stores the same in a database at a backend server (not illustrated in the figure). The first type 106 of tagged writing surface includes an addendum code 110, and the second type 108 of tagged writing surface includes a unique first code 112. In one embodiment, the codes 110 and 112 are linked to patient identity and other patient particulars so that the corresponding portion of the tagged writing surface is easily shareable and searchable in the database. In one embodiment herein, the doctor 102 possesses an electronic method of data entry such as HIS/EMR/EHR etc. and usually prints out the data on the tagged writing surfaces to be handed over to patients. In this scenario, a virtual printer driver runs on a computing device of the doctor that may accomplish: a) sharing of the digital (to be printed) data with a backend server; b) generating a code printing on the tagged writing surface. Later on, anyone with relevant permissions can click on the codes 110, 112 to get access to the original printed data. Note here that anything that the doctor 102 writes with the tagged writing surface may not be available to the accessing patient. In another embodiment herein, pre-printed tagged writing surfaces are used by assistants 114 for filling the doctor's data.

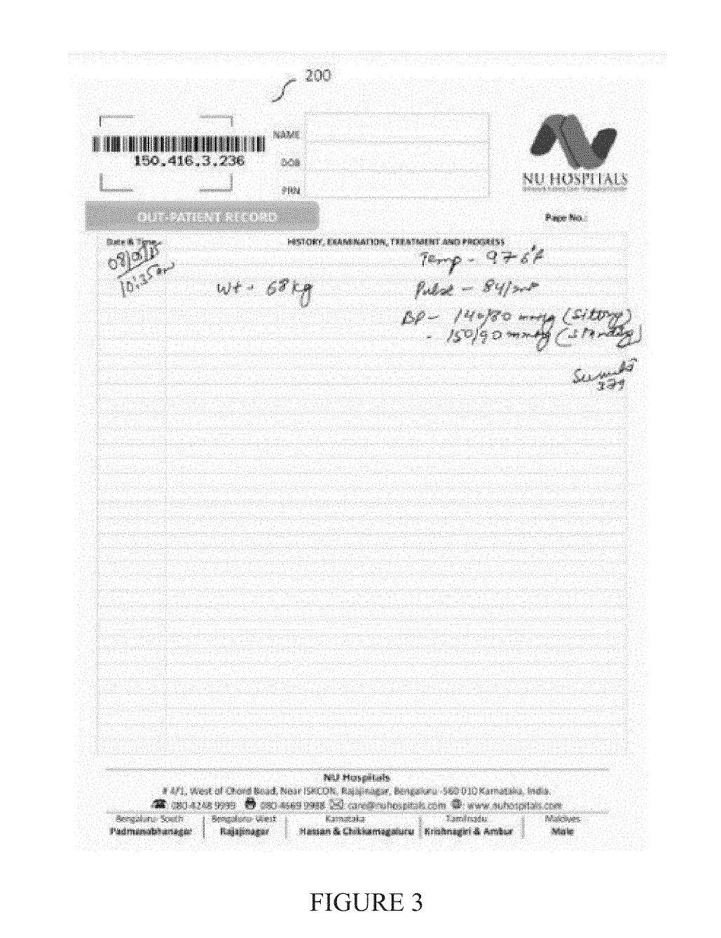

FIG. 3 illustrates a sample tagged writing surface with a code at the top. Each tagged writing surface 200 is preprinted with a code that has a unique identity to identify the tagged writing surface. While generating a second code or an addendum code, inputs from the first code are also input to assist in clinical workflow. The data comprises inputs such as PRN (Patient Registration No.), Name, Age, Contact Details, and Medical History information to generate a unique code.

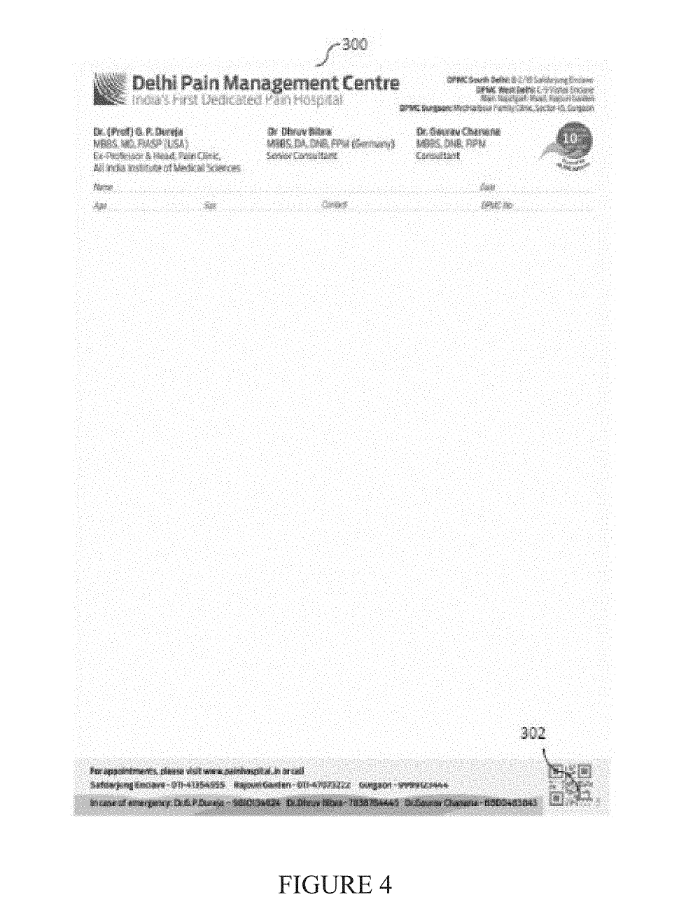

FIG. 4 illustrates a sample tagged writing surface with a pre-printed code at the bottom. Each paper in this case is pre-printed with a customized patient particular code on top of the paper 300. The fields such as Name, Age, and Sex are transcribed and tagged with a particular first code. In this scenario, the tagged writing apparatus provides the handwritten data (ink) on the paper for integration with the backend server. At a later stage, the code is scanned by a relevant entity or node of the network to get access to the original data.

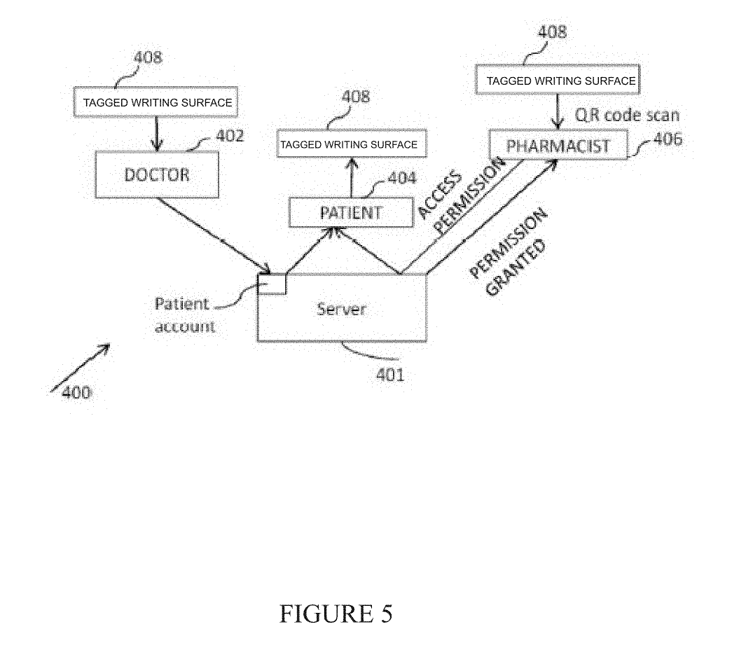

FIG. 5 illustrates a platform for data sharing and consumption. The online platform 400 is hosted on a cloud and care providers such as doctors, pharmacists, hospitals, and laboratories register for relevant patient data access. The platform 400 includes a server 401 that provides a healthcare application for execution on at least the computing devices of the doctor 402, patient 404, pharmacist 406. Examples of the computing devices, include, but are not limited to, smart phones and tablets. In one embodiment, a doctor 402 sees a patient 404 at a clinic or a hospital, and gives a hard copy of the tagged writing surface 408 to the patient. The tagged writing surface (both preprinted and handwritten) may be saved at the server 401 and can be shared among the doctor 402, patient 404, and pharmacist 406 via the application or other online access means. In another embodiment, a doctor 402 consulting a patient 404 via phone or online methods may keep the hard copy of the tagged writing surface 408 with himself/herself, and provide a soft copy of the tagged writing surface 408 (also known as tele-prescription) to the patient 404 via corresponding application. In yet another embodiment, if any other node or entity requires access to the original data, a request first reaches the server (administrator) 401, where a rule engine determines if defined rules allow for such access/sharing, and a count is maintained on who (doctors, pharmacists, hospitals, laboratories) is accessing the data and at what date and time. An access permission may be sought from the patient 404 and then the soft copy may be served by the server 401. Thus, the access is controlled at the server 401 about who can access based on their clinical needs and profiles. In yet another embodiment, the server 401 may add value to the process by ‘tagging’ the forms and performing medical transcription so that when documents appear on the doctor's and patient's computing devices, they are easily searched.

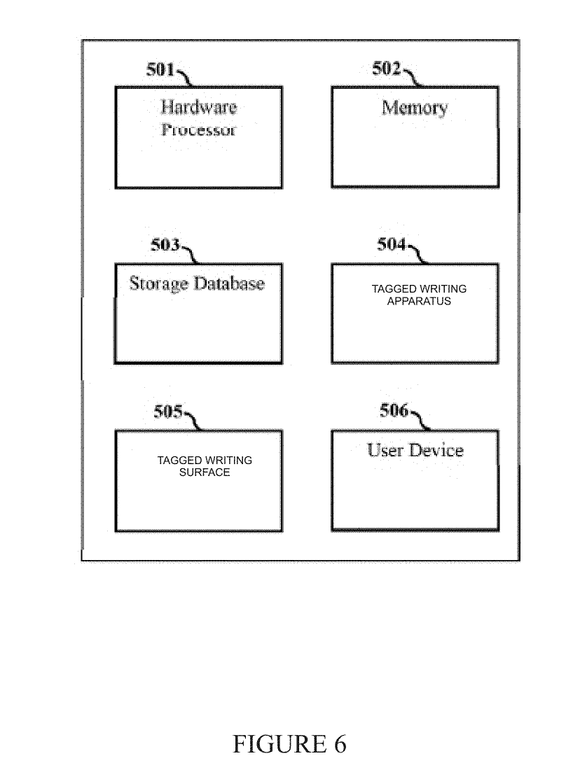

FIG. 6 illustrates a system for data sharing and consumption in a healthcare information management system using the tagged writing surface and the tagged writing apparatus. The system comprises a hardware processor 501, a memory 502, a storage database 503, a tagged writing apparatus 504, a tagged writing surface 505 and a user-device 506.

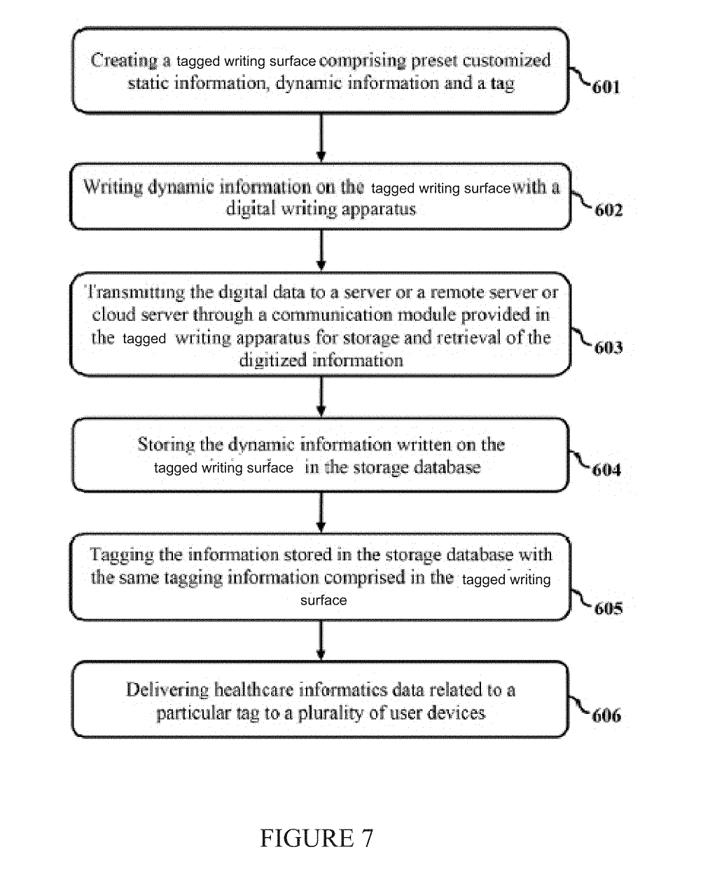

FIG. 7 illustrates a flow chart explaining a process for data sharing and consumption in a healthcare information management system using the tagged writing apparatus and the tagged writing surface. The process comprises the following steps: creating a prescription sheet comprising preset customized static information, dynamic information, and a tag (601); writing dynamic information on the tagged writing surface with a tagged writing apparatus (602); transmitting the digital data to a server or a remote server or cloud server through a communication module provided in the tagged writing apparatus for storage and retrieval of the digitized information (603); storing the dynamic information written on the tagged writing surface in the storage database (604); tagging the information stored in the storage database with the same tagging information comprised in the tagged writing surface (605); and, delivering healthcare informatics data related to a particular tag to a plurality of user devices (606).

In at least an embodiment, for certain use cases such as insurance matters, a wet-sign of a doctor is a final authenticator for a claim to be processed. In prior art, print outs are taken after which a doctor wet signs the print out, scans it, and then OCR is performed for data to be scanned. With the use of this system and method, the process of OCR is eliminated since virtual drivers ensure that, in the connected node environment, data is already transmitted to the insurance node in a secure and masked manner and this data is only unmasked with a doctor wet-signs an associated printed-out matter of the doctor-patient interaction captured on the tagged writing surface by means of a tagged writing apparatus.

In another embodiment, even print outs can be eliminated. For each doctor, for each signature, identification vectors may be stored based on strokes and such signature-identification criteria. When the doctor, whose identification vectors are stored, performs his or her signature on the tagged writing surface, by means of the tagged writing apparatus, the identification vectors are captured and compared, by a comparator, with stored signature-identification criteria so as to ensure authenticity. Upon authentication, rule based actions may be performed by the system and method of this invention.

The need to OCR has been eliminated; because all data associated with doctor and patient per first code and per second code is already stored in databases. The wet signature merely allows that the doctor has authorized an insurance agent to read it.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the disclosure with modifications. However, all such modifications are deemed to be within the scope of the appended claims.

It is also to be understood that the following claims are intended to cover all of the generic and specific features of the embodiments described herein and all the statements of the scope of the embodiments which as a matter of language might be the to fall there between.

What is claimed is:

1. A system for storing and delivering healthcare informatics data within a network of nodes, the system comprising:
a tagged writing receiver having a first code that provides an identity to the tagged writing receiver, wherein the tagged writing receiver is configured to have content written on it in real-time and transmitted to a digital medium in real-time, wherein the tagged writing receiver has an addendum code that is activatable only by activation of a sub-zone of the tagged writing receiver, and wherein the addendum code when activated causes the content that is in the sub-zone in to be stored in an access-controlled database associated with a node in the network; and
a tagged writing instrument configured to write the content on the tagged writing receiver and concurrently capture the content from the tagged writing receiver, wherein the tagged writing instrument has a second code that provides the identity to the tagged writing instrument, and wherein the tagged writing instrument is configured to convert at least some of the content into recognized data items.

2. The system of claim 1, wherein the identity is at least one of an operator and owner of the tagged writing receiver or the tagged writing instrument, and wherein the first code and the second code are stored in separate databases.

3. The system of claim 1, wherein the sub-zone is one of a plurality sub-zones made up of pixels on the tagged writing receiver, and wherein the tagged writing instrument identifies the sub-zone and activates the addendum code based on contact with the sub-zone.

4. The system of claim 1, wherein the tagged writing instrument is configured to identify digital data items from the content, and wherein the addendum code when activated causes the tagged writing instrument to handle the digital data items according to a rule engine associated with the sub-zone.

5. The system of claim 4, wherein the tagged writing instrument includes an imaging device configured to convert the content into the digital data items and transmit the data items to the network of nodes.

6. The system of claim 4, further comprising:
a tagging station configured to tag on a per clinic or per hospital or per doctor basis by simultaneously reading the first code and at least one of the addendum code and the second code and associating the read codes with each other or with identities, wherein the tagging station is associated with the rule engine to define rules for each association.

7. The system of claim 1, wherein the first code correlates with a set of dynamic information written on the tagged writing receiver including at least one of a patient's name, patient's demographics, patient's identity, patient's illness, patient's treatment plan, prescription, date of doctor-patient interaction, time of doctor-patient interaction, and location of doctor-patient interaction.

8. The system of claim 1, wherein the tagged writing receiver is further configured to correlate at least one of a doctor identity with the first code and a corresponding tagged writing receiver, a doctor identity with the second code and a corresponding tagged writing instrument, a patient identity with the first code and a corresponding tagged writing receiver, a patient identity with the second code and a corresponding tagged writing instrument, a hospital identity or a clinic identity with the first code and a corresponding tagged writing receiver, and a hospital identity or a clinic identity with the second code and a corresponding tagged writing instrument.

9. The system of claim 1, wherein the tagged writing instrument is further configured to weight and map pixels on the tagged writing receiver by absolute position on the tagged writing receiver and relative position to the sub-zone of the tagged writing receiver, wherein a weight of each pixel defines an origin node associated with that pixel and a destination node associated with that pixel so as to make the tagged writing receiver dynamic.

10. The system of claim 1, wherein the sub-zone on the tagged writing receiver is contoured or outlined, further comprising:
a display configured to display a pixel weight and map within a contour of the sub-zone and configured to receive a user association of the pixel with the addendum code to define an origin or destination node of the pixel of the sub-zone

11. The system of claim 1, further comprising:
a comparator configured to identify a doctor's signature in real time on the tagged writing receiver from identification vectors, wherein identifying the doctor's signature authenticates the content on the tagged writing receiver.

12. A method of storing and delivering healthcare informatics data within a network of nodes, the system comprising:
providing a tagged writing receiver having a first code that provides an identity to the tagged writing receiver, wherein the tagged writing receiver is configured to have content written on it in real-time and transmitted to a digital medium in real-time, wherein the tagged writing receiver has an addendum code that is activatable only by activation of a sub-zone of the tagged writing receiver, and wherein the addendum code when activated causes the content that is in the sub-zone in to be stored in an access-controlled database associated with a node in the network; and
providing a tagged writing instrument configured to write the content on the tagged writing receiver and concurrently capture the content from the tagged writing receiver, wherein the tagged writing instrument has a second code that provides the identity to the tagged writing instrument, and wherein the tagged writing instrument is configured to convert at least some of the content into recognized data items.

13. The method of claim 12, wherein the identity is at least one of an operator and owner of the tagged writing receiver or the tagged writing instrument, and wherein the first code and the second code are stored in separate databases.

14. The method of claim 12, wherein the sub-zone is one of a plurality sub-zones made up of pixels on the tagged writing receiver, and wherein the tagged writing instrument identifies the sub-zone and activates the addendum code based on contact with the sub-zone.

15. The method of claim 12, wherein the tagged writing instrument is configured to identify digital data items from the content, and wherein the addendum code when activated causes the tagged writing instrument to handle the digital data items according to a rule engine associated with the sub-zone.

16. The method of claim 15, wherein the tagged writing instrument includes an imaging device configured to convert the content into the digital data items and transmit the data items to the network of nodes.

17. The method of claim 12, further comprising:
providing a tagging station configured to tag on a per clinic or per hospital or per doctor basis by simultaneously reading the first code and at least one of the addendum code and the second code and associating the read codes with each other or with identities, wherein the tagging station is associated with the rule engine to define rules for each association.

18. The method of claim 12, wherein the first code correlates with a set of dynamic information written on the tagged writing receiver including at least one of a patient's name, patient's demographics, patient's identity, patient's illness, patient's treatment plan, prescription, date of doctor-patient interaction, time of doctor-patient interaction, and location of doctor-patient interaction.

19. The method of claim 12, wherein the tagged writing receiver is further configured to correlate at least one of a doctor identity with the first code and a corresponding tagged writing receiver, a doctor identity with the second code and a corresponding tagged writing instrument, a patient identity with the first code and a corresponding tagged writing receiver, a patient identity with the second code and a corresponding tagged writing instrument, a hospital identity or a clinic identity with the first code and a corresponding tagged writing receiver, and a hospital identity or a clinic identity with the second code and a corresponding tagged writing instrument.

20. The method of claim 12, wherein the sub-zone on the tagged writing receiver is contoured or outlined, further comprising:
providing a display configured to display a pixel weight and map within a contour of the sub-zone and configured to receive a user association of the pixel with the addendum code to define an origin or destination node of the pixel of the sub-zone; and
providing a comparator configured to identify a doctor's signature in real time on the tagged writing receiver from identification vectors, wherein identifying the doctor's signature authenticates the content on the tagged writing receiver.
